(19) United States
(12) Patent Application Publication
(10) Pub. No.: US 2004/0213703 A1
(43) Pub. Date: Oct. 28, 2004

(54) Multi-lamp germicidal treatment system with interlock

(75) Inventors: Robert M. Culbert, Manhattan Beach, CA (US); Kanghong Zhang, Long Beach, CA (US)

Correspondence Address: SOCAL IP LAW GROUP, 310 N. WESTLAKE BLVD. STE 120, WESTLAKE VILLAGE, CA, 91362, US

(73) Assignee: Steril-Aire USA, Inc.

(21) Appl. No.: 10/423,573

(22) Filed: Apr. 24, 2003

(51) Int. Cl. A61L 9/20

(52) U.S. Cl. 422/121

(57) ABSTRACT

There is disclosed a multi-lamp germicidal treatment system with interlock. The multi-lamp germicidal treatment system includes two or more mounting assemblies, one of which is coupled to a power source. The mounting assemblies are adjustably coupled to one another. A germicidal lamp may be coupled to each of the mounting assemblies. Power is only provided to each of the germicidal lamps when all of the mounting assemblies have a germicidal lamp coupled therein.

### NOTICE OF COPYRIGHTS AND TRADE DRESS

A portion of the disclosure of this patent document contains material which is subject to copyright protection. This patent document may show and/or describe matter which is or may become trade dress of the owner. The copyright and trade dress owner has no objection to the facsimile reproduction by any one of the patent disclosure as it appears in the Patent and Trademark Office patent files or records, but otherwise reserves all copyright and trade dress rights whatsoever.

### BACKGROUND OF THE INVENTION

1. Field of The Invention

The invention relates to germicidal treatment systems that include germicidal lamps.

2. Description of Related Art

Heating, ventilation and air conditioning (HVAC) units may be augmented with germicidal systems that emit ultraviolet light to eradicate harmful microorganisms in the air, in ducts and on and in HVAC units. Typical germicidal systems comprise a lamp and a fixture. The fixture typically includes an electronic or magnetic ballast. Germicidal systems may have one or more lamps. The installation of multiple germicidal systems and/or multiple lamp germicidal systems may be required to obtain effective coverage and treatment.

The UV radiation emitted by the germicidal lamps in germicidal systems is dangerous to the human eye. When installing and maintaining germicidal systems, it is imperative that the user not be exposed to the UV light emitted from the germicidal systems. When replacing germicidal lamps in multiple lamp germicidal systems and installing multiple lamp germicidal systems, it is important that the user not irradiate a first lamp while installing a second lamp, and vice versa.

### DESCRIPTION OF THE DRAWINGS

The invention will be described by way of exemplary embodiments, but not limitations, illustrated in the accompanying drawings in which like references denote similar

FIG. 1 is a partially exploded perspective view of a dual-lamp germicidal treatment system having an integrated lamp.

FIG. 2 is a top perspective view of a dual-lamp germicidal treatment system attached to a wall.

FIG. 3 is an exploded perspective view of a mounting assembly of a dual-lamp germicidal treatment system.

FIG. 4 is a bottom view of a circular coordinated lamp connector and circular coordinated receivers.

FIG. 5 is a bottom view of a square coordinated lamp connector and square coordinated receivers.

FIG. 6 is schematic diagram of an embodiment of the circuitry included in a coordinated connector.

FIG. 7 is a schematic diagram of an embodiment of the interlock circuit included in the coordinated receivers.

FIG. 8 is a block diagram of an embodiment of a completed interlock circuit formed when germicidal lamps are included in the mounting assemblies of a germicidal treatment system.

### DETAILED DESCRIPTION OF THE INVENTION

Throughout this description, the embodiments and examples shown should be considered as exemplars, rather than limitations on the apparatus and methods of the invention.

As used herein, the term air handling apparatus includes air carrying ducts and air treating units, such as heating, ventilation and air conditioning (HVAC) units of all kinds including, but not limited to, air conditioners, heaters, humidifiers, and dehumidifiers, whether installed as a single unit, as multiple units and in combination, as well as any air treatment or air delivery device and associated ducts.

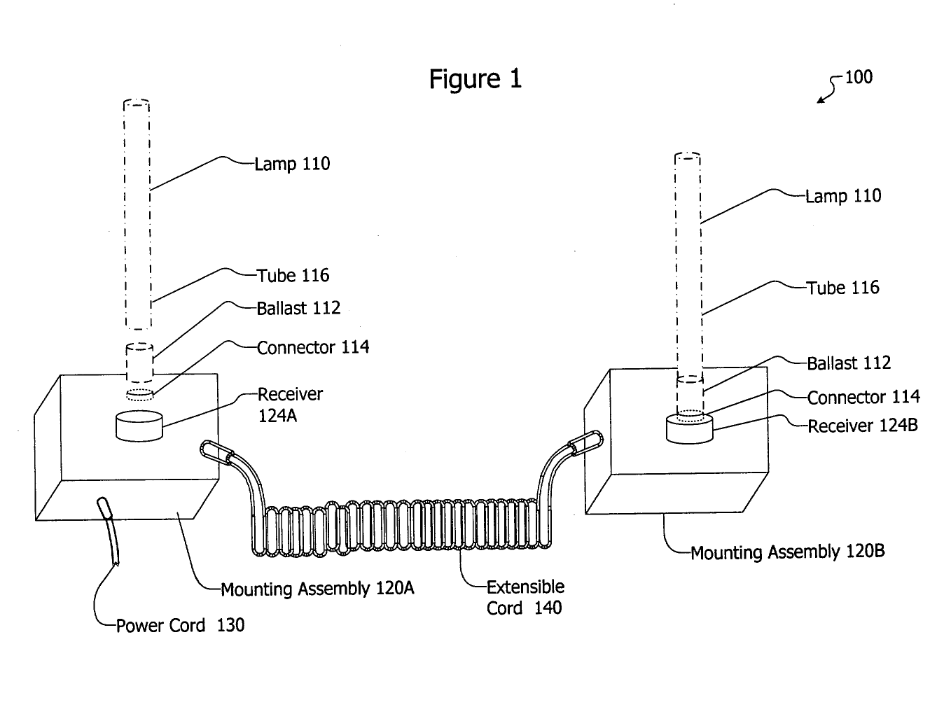

FIG. 1 is a partially exploded perspective view of a dual-lamp germicidal treatment system 100 having integrated lamps. In this embodiment, germicidal treatment system 100 includes two or more germicidal lamps 110 which are coupled to mounting assembly 120A and mounting assembly 120B. Mounting assembly 120A and mounting assembly 120B are adjustably coupled together with a extensible coiled cord 140.

In one embodiment, the germicidal lamps 110 include a ballast 112, a coordinated connector 114, and a tube 116. As used herein, the term integrated lamp refers to those germicidal lamps that include a ballast, a coordinated connector, and a tube. In another embodiment, the germicidal lamp 110 may be comprised of a tube 116 and a coordinated connector 114. The tubes 116 may have a T5, T8, or other diameter.

In one embodiment, the germicidal lamps 110 are single ended. Single tube and multiple tube single ended germicidal lamps as well as u-shaped, j-shaped and other shaped single ended germicidal lamps may be incorporated in the germicidal treatment systems described herein. In other embodiments, the germicidal lamps may be double ended. The germicidal lamps 110 may have any appropriate length, such as, for example, without limitation, 12″, 16″, 20″, 24″, 30″, 36″, 40″, 48″, 56″and 64″.

The germicidal lamps 110 may be those germicidal lamps that emit sufficient UV-C radiation to eradicate airborne germs and germs that accumulate in the air in and on the internal surfaces of air handling apparatus. In one embodiment, the lamps 110 emit broadband UV-C in the range of 250-260 nm.

The ballast 112 is coupled between the tube 116 and the connector 114. The ballast 112 may receive electrical power from connector 114 when the integrated lamp 110 is placed in receiver 124A. The ballast 112 may be any electrical or magnetic ballast sufficient to control the electrical power that will be provided to the tube 116. In one embodiment, ballast 112 is round. In various embodiments, ballast 112 may have generally the same diameter as the diameter of tube 116, may have a diameter greater than that of tube 116, or may have a diameter smaller than the diameter of tube 116. In other embodiments, the ballast 112 is not round and may be any shape.

Coordinated connector 114 is used to couple the germicidal lamp 110 to the mounting assemblies 120A and 120B. Coordinated connector 114 may include a plastic, resin, ceramic or other nonconductive base having metal prongs, strips or other conductive portion. The metal prongs, strips or other conductive portion is constructed to receive electrical current from receivers 124A and 124B in the mounting assemblies 120A and 120B. Coordinated connector 114 is designed to correspond to the opening provided in receivers 124A and 124B of the mounting assemblies 120A and 120B.

The receivers 124A and 124B are coupled to the mounting assemblies 120A and 120B. The receivers 124A and 124B are a coordinated receiving sockets that correspond to the matching coordinated connector 114 included with the germicidal lamps 110. The receivers 124A and 124B may include a nonconductive portion and a conductive portion. The conductive portion may be metal or be any other conductive material. The nonconductive portion may include a plastic, resin, ceramic or other nonconductive sleeve. The conductive portion is constructed to pass electrical current from the mounting assemblies 120A and 120B to the germicidal lamps 110.

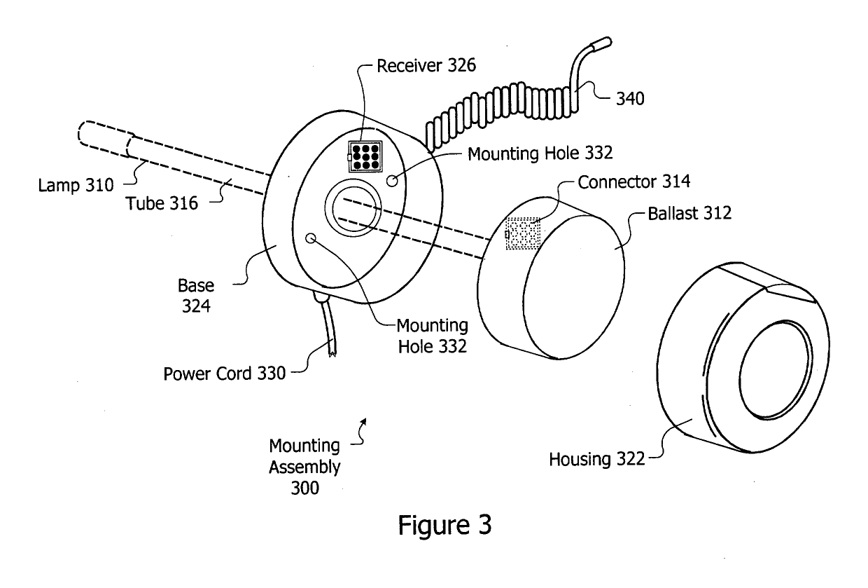

Example configurations of the coordinated receivers 124A and 124B and connector 114 are discussed in more detail below with regard to FIG. 3.

Mounting assemblies 120A and 120B may be manufactured from any resilient, strong, durable material that is impervious to temperature changes, such as, for example, but not limited to, metals, plastics, and resins, unless otherwise specified. In some embodiments, the exterior of mounting assemblies 120A and 120B is impervious to water so that the contents of the housing remain dry. The mounting assemblies 120A and 120B may be any shape such as generally rectangular, square, circular, etc. The mounting assemblies 120A and 120B are adjustably coupled to one another via extensible coiled cord 140.

Extensible coiled cord 140 is coupled on one end to mounting assembly 120A and on the other end to mounting assembly 120B. Extensible coiled cord 140 allows the adjustable germicidal treatment system 100 to be installed on the walls of various configurations of air handling apparatus. The extensible coiled cord 140 allows for any desired placement of the germicidal lamps 110 when the germicidal treatment system 100 is attached on a wall of an air handling apparatus.

In addition, extensible coiled cord 140 passes electrical power between mounting assemblies 120A and 120B. Extensible coiled cord 140 serves as a power channel, power line or other power carrying medium.

In other embodiments, the extensible coiled cord 140 may be replaced with any coupling apparatus that allows the mounting assemblies to be adjusted, such as for example, arms with hinges, accordion portions, telescoping portions, interlocking portions, and others.

Power cord 130 is coupled to mounting assembly 120A to provide electrical power to the germicidal treatment system 100. That is, power cord 130, which is only coupled to one of the mounting assemblies 120A of the germicidal treatment system 100, provides power to each of the mounting assemblies 120A and 120B.

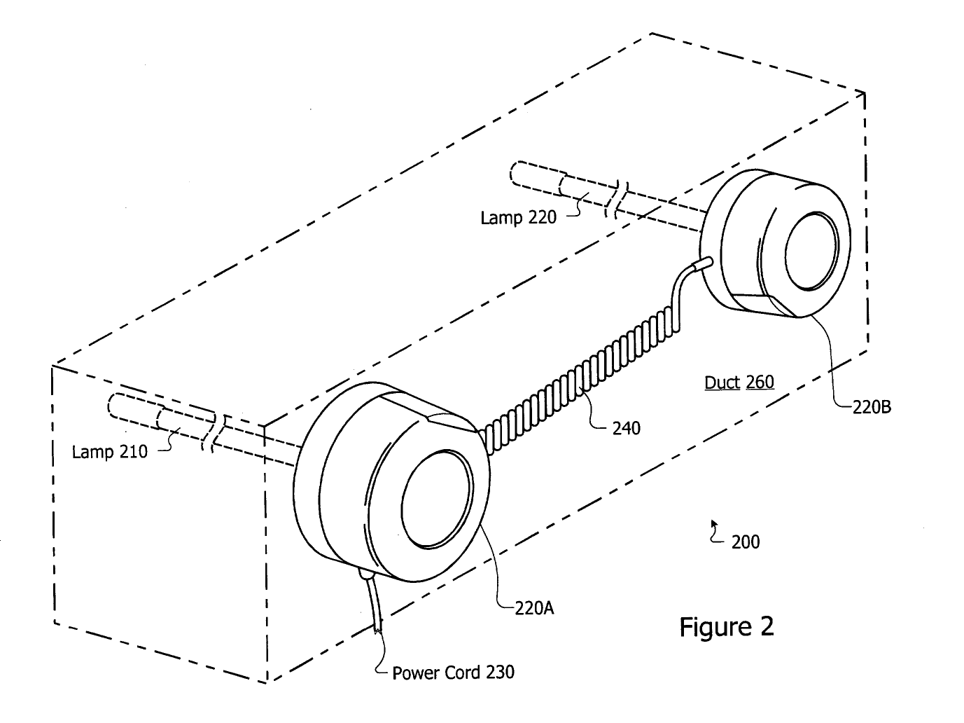

FIG. 2 is a top perspective view of dual-lamp germicidal treatment system 200 attached to duct wall 260. As shown, the mounting assemblies 220A and 220B are generally circular in shape and are coupled together with extensible cord 240. The germicidal treatment shape receives power from power cord 230. In various embodiments, the mounting assemblies 220A and 220B may include receivers that are flush to the mounting assemblies and sit flush to the wall of an air handling apparatus when attached, or the receivers may extend from the mounting assemblies through the wall of an air handling apparatus.

More specifically, in one embodiment, when the germicidal treatment system 200 is installed on duct wall 260, the receivers (not shown) of the mounting assemblies 220A and 220B are situated on an outside side of the duct wall 260 of an air handling apparatus. The lamps 210 pass through a hole in the duct wall 260 from an inside side of the duct wall 260 through the duct wall 260 and are coupled to the receivers of mounting assemblies 220A and 220B on the outside side of the duct wall 260. In this embodiment, the receiving holes have a diameter sufficient to accommodate the lamps 210.

In another embodiment, the receivers extend from the mounting assemblies 220A and 220B. In this embodiment, when the germicidal treatment system 200 is installed on duct wall 260, the mounting assemblies 220A and 220B are situated on an outside side of a duct wall 260 and pass through the receiving hole to an inside side of the duct wall 260. In this embodiment, the receiving holes are of a diameter sufficient to accommodate the receivers.

The mounting assemblies 220A and 220B of the germicidal treatment system 200 may be attached to the wall of an air handling apparatus using screws, bolts, rivets, glue, and other fasteners. In other embodiments, hook and loop fasteners such as Velcro® brand hook and loop fasteners may be used to attach the germicidal treatment system to the wall of an air handling apparatus.

FIG. 3 is a perspective view of a mounting assembly of a dual-lamp germicidal treatment system. Mounting assembly 300 includes a base 324 into which a germicidal lamp 310 is inserted. Mounting assembly 300 includes a housing 322 to be placed over the germicidal lamp 310 when it is attached to base 324.

Base 324 may be coupled to a wall or housing of an air handling apparatus as shown for example, in FIG. 2, via bolts, screws, rivets or other fasting devices placed through mounting holes 332. Although two mounting holes 332 are shown, one or more mounting holes may be included in base 324. Base 324 may include power cord 330 which may be coupled to a power source. Base 324 may include extensible cord 340 by which the mounting assembly 300 is coupled to at least one, paired mounting assembly. In one embodiment, the mounting assemblies 300 may be daisy chained into a string of two or more mounting assemblies. Base 324 also includes receiver 326 to which a coordinated connector 314 of germicidal lamp 310 is coupled. Base 324 includes a hole of sufficient diameter through which the tube portion 316 of germicidal lamp 310 may pass.

Germicidal lamp 310 may be an integrated lamp that includes ballast portion 312 and tube 316. In one embodiment, the ballast portion includes connector 314 which corresponds to receiver 326. Germicidal lamp 310 is placed in base 324 and may be held in place by coupling connector 314 with receiver 326. In some embodiments, other alignment protrusions and receivers may be included in the base 324 and the ballast portion 312 of germicidal lamp 310.

Housing 322 is placed over the ballast portion 312 of germicidal lamp 310 and couples to base 324. The housing 322 and base 324 may have corresponding receivers and protrusions to allow for easy attachment and later removal of housing 322, such by snapping on and off.

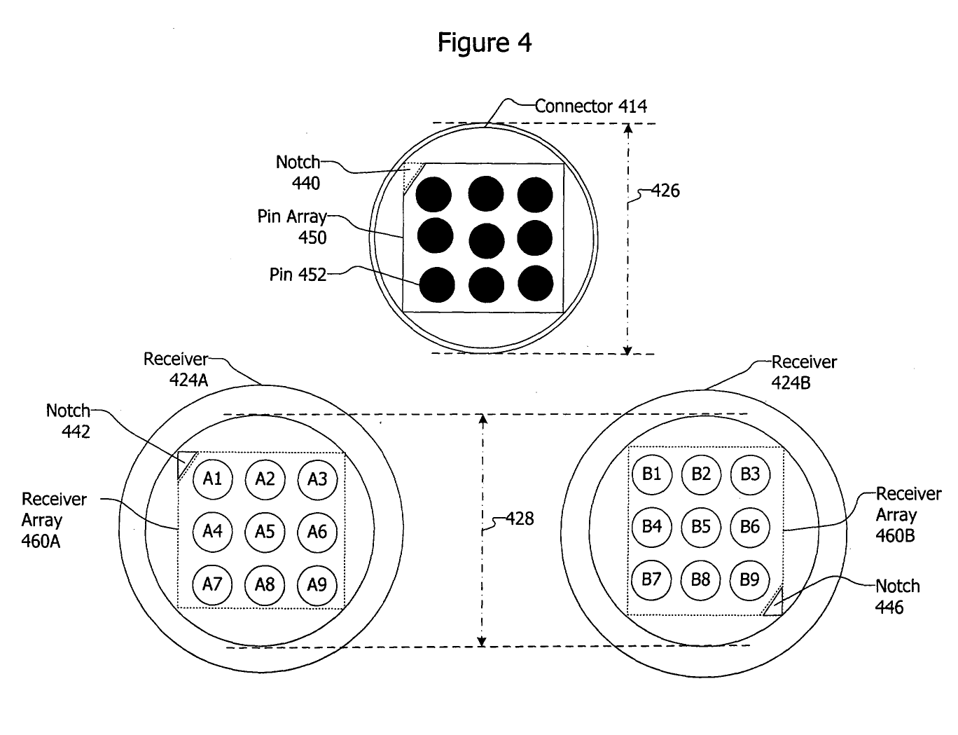

FIG. 4 is a bottom view of circular coordinated lamp connector 414 and circular coordinated receivers 424A and 424B. These may be lamp connector 114 and coordinated receivers 124A and 124B of FIG. 1. In one embodiment, connector 414 is generally circular and includes a square pin array 450 of multiple pins 452. In other embodiments, the connector 414 may be any shape. Connector 414 has diameter 426 which generally corresponds to the diameter of ballast 112. The areas of connector 414 are made of one or more non-conductive materials such as, for example, plastic or ceramic, except for the pins 452. The pins 452 are made of metal or other conductive material to pass electrical current to the ballast of a germicidal lamp.

In one embodiment, the pin array 450 includes nine pins 452 arranged in a symmetrical square. In other embodiments, the shape of the pin array 450 may be circular or any shape. The number of pins 450 corresponds to the number of receiving holes in coordinated receivers. The number of pins may be as few as three and as many as is physically practicable. In one embodiment, the pin array 450 includes a notch or other male coordinating element such as, for example, triangular notch 440, which aligns germicidal lamps in receivers 424A and 424B. The male coordinating element in the connector 414 may be considered a protrusion. The male coordinating element may be any shape. The particular shape of the male coordinating element is not pertinent. What is pertinent is that the male coordinating element in the connectors 414 has a corresponding female coordinating element in the receivers 424A and 424B. In another embodiment, the receivers have a male coordinating element and the connectors have a female coordinating connector.

Receivers 424A and 424B are generally circular and have an internal diameter 428 large enough to receive connector 414. The internal diameter 428 generally corresponds to the diameter 426 of connector 414. Similarly, receiver arrays 460A and 460B are generally a size sufficient to receive pin array 450 of connector 414. Receiver arrays 460A and 460B each have nine receiving holes A1-A9 and B1-B9, respectively. The number of receiving holes corresponds to the number of pins 452 in pin array 450. As with the connector 414 and the pin array 450, in other embodiments, the receivers 424A and 424B and the receiver arrays 460A and 460B may be any shape.

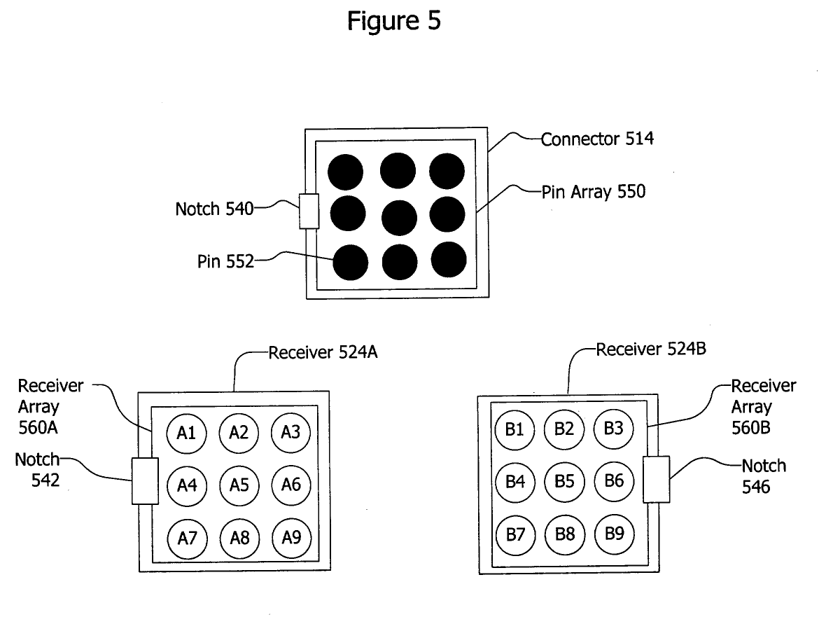

FIG. 5 is a bottom view of a square coordinated lamp connector and square coordinated receivers. These may be connector 314 and coordinated receiver 326 of FIG. 3. In one embodiment, connector 514 is generally square and includes a square pin array 550 of multiple pins 552. The areas of connector 514 are made of one or more non-conductive materials such as, for example, plastic or ceramic, except for the pins 552. The pins 552 are made of metal or other conductive material to pass electrical current to the ballast in the germicidal lamp.

In one embodiment, the pin array 550 includes nine pins 552 arranged in a square three by three array. In other embodiments, the shape of the pin array 550 may be any shape. The number of pins 550 corresponds to the number of receiving holes in corresponding coordinated receivers. The number of pins may be as few as three and may be as many as is physically practicable.

In one embodiment, the pin array 550 includes a notch or other male coordinating element such as, for example, notch 540, which aligns germicidal lamps in receivers, such as, for example, referring to FIG. 4, connector 314 of germicidal lamp 310 in receiver 326. The male coordinating element in the connector 514 may be considered a protrusion. The male coordinating element may be any shape. The particular shape of the male coordinating element is not pertinent. What is pertinent is that the male coordinating element in the connector 514 has a corresponding female coordinating element in the receivers 524A and 524B. In another embodiment, the receivers have a male coordinating element and the connectors have a female coordinating connector. In other embodiments, the connectors and receivers may each have two or more male and female coordinating elements.

Receivers 524A and 524B are generally square and have a size large enough to receive connector 514. Similarly, receiver arrays 560A and 560B are generally the same size as pin array 550 of connector 514. Receiver arrays 560A and 560B each have nine receiving holes A1-A9 and B1-B9, respectively. The number of receiving holes corresponds to the number of pins 552 in pin array 550.

In another embodiment, the receiver arrays and the connector arrays may be generally flat and sit flush against each other. In this embodiment, the coordinating elements of the connector and receiver align the receiver and the connector together. A key and hole technique may be employed. The remaining portions of the receiver and the connector serve to attach the receiver and the connector together, as described herein.

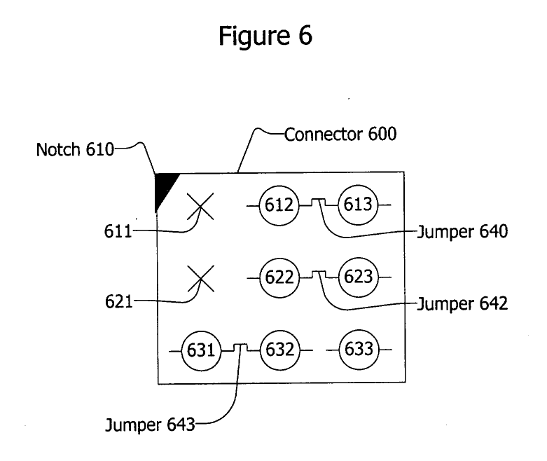

FIG. 6 is schematic diagram of an embodiment of the circuitry included in a coordinated connector. Each of the lamps described herein have a coordinated connector that includes an embodiment of the circuitry shown in connector 600. In one embodiment, each of the connectors in each of the lamps are the same. In another embodiment, the lamps may have different connectors, the connector being dependent on whether the lamp is being coupled to a mounting assembly which is coupled to a power source, or a mounting assembly coupled to one or mounting assemblies while not being coupled to a power source. Connector 600 is used to complete the electrical circuit between the mounting assemblies when placed in the coordinated receivers included in the mounting assemblies. Connector 600 shows the circuitry included in the connectors 414 and 514 described above.

Connector 600 includes notch 610 which aligns the connector 600 with the receivers shown in the other drawings described herein. Notch 610 may be used to key the connection between the connector and the receivers. In other embodiments, in place of or in addition to the notch 610, the pin heights on connectors may vary to correspond to varying receiver hole depth to allow for aligning and keying. In various embodiments, one or more male and/or female coordinating elements are included in connector 600 which correspond to corresponding female and/or male elements included in receivers.

In other embodiments, a snap-in or clip-in attaching technique may be incorporated such that a connector notch pushes the receiving notch away with the application of a modicum of force so that the connecting notch passes by the receiving notch and the receiving notch snaps or moves back into place such that the connector notch is located behind the receiving notch. In these embodiments, the receiver and the connector may be comprised of, at least in part, a sufficiently flexible rigid material such as plastic.

Connector 600 comprises an array of terminals and one or more jumpers. The array of terminals may include active terminals and unused terminals. The array of terminals corresponds to the pin arrays 450 and 550 shown in FIGS. 4 and 5. When the connector 600 is coupled with receivers, the active terminals and jumpers complete the interlock circuit between the mounting assemblies of and provide power to the lamps in a germicidal treatment device.

In one embodiment, connector 600 includes a three by three array of nine terminals. The terminals may be referred to by their array locations, with the top left terminal being 11 and the bottom right terminal being 33. To ease understanding of the description provided herein, the terminals are labeled to reflect their location pre-pended with the number 6. In one embodiment, two terminals, terminals 611 and 621, are unused terminals. Terminals 612, 613, 622, 623, 631, 632 and 633 are active terminals. Jumper 640 couples terminals 612 and 613, jumper 642 couples terminals 622 and 623, and jumper 643 couples terminals 631 and 632.

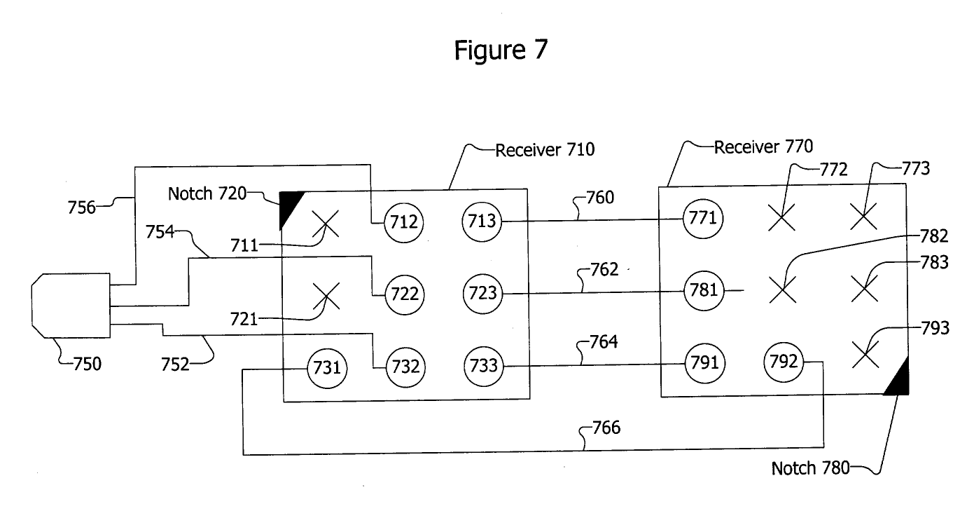

FIG. 7 is a schematic diagram of an embodiment of the interlock circuit included in the coordinated receivers. Receiver 710 comprises a three by three array of terminals which includes active terminals and unused terminals. In one embodiment, terminals 711 and 721 are unused terminals, and terminals 712, 713, 722, 723, 731, 732, and 733 are active terminals.

Receiver 710 includes notch 720 which allows the receiver 710 to be coupled in a coordinated manner with a connector such as connector 600.

Receiver 710 receives electrical power from a power source 750. The power is distributed to the terminals by lines 756, 754 and 752. In one embodiment, line 754 may be a ground, and lines 752 and 756 may carry electrical current. Lines 752 and 756 may be considered “hot.” Line 752 couples the power source 750 to terminal 732, line 754 couples the power source 750 to terminal 722, and line 756 couples the power source 756 to terminal 712. In one embodiment, line 752 may be black, line 754 may be green, and line 756 may be white. As shown in FIG. 7, there are no connections between active terminals 712 and 713, between active terminals 722 and 723, between active terminals 732 and 733, and between active terminals 732 and 731. As such, electrical power received on line 752 terminates at terminal 732 and is not passed to terminal 731. Similarly, electrical power received on line 756 terminates at terminal 712 and is not passed to terminal 713.

Receiver 770 comprises a three by three array of terminals which include active terminals and unused terminals. In one embodiment, terminals 772, 773, 782, 783, 791, and 793 are unused terminals, and terminals 771, 781, 791 and 792 are active terminals.

Receiver 770 includes notch 780 which allows the receiver 770 to be coupled in a coordinated manner with a connector such as connector 600. From one perspective, the location of notch 780 is in a location different from the location of the notch 720 of receiver 710. In receiver 770, the notch 780 is at the bottom right of the receiver 770 adjacent to terminal 733, whereas in receiver 710 the notch 720 is located in the top left corner of receiver 710 adjacent to terminal 711. The location of the notches 720 and 780 differ so that the receivers may receive the connector 600 to achieve the interlock described herein. From another perspective, the notches exist in both receivers in the same place, but the underlying circuitry, that is, the number and arrangement of unused terminals and active terminals, differs between the receivers 710 and 770.

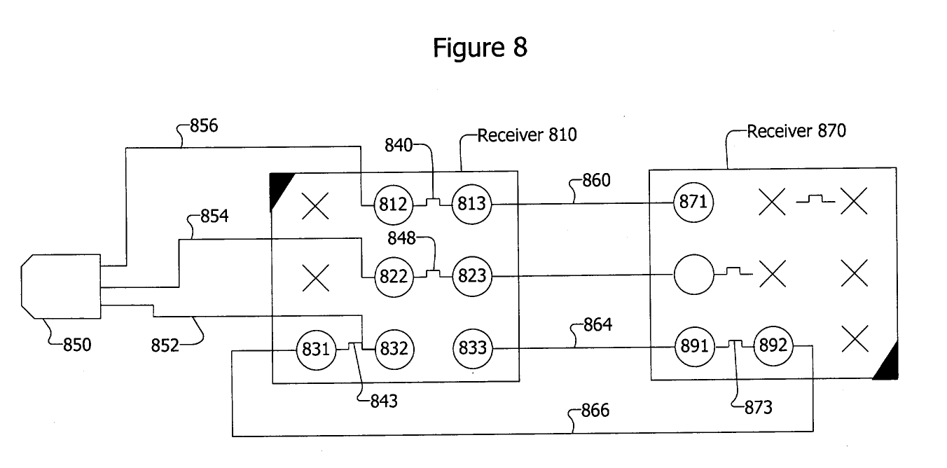

Lines 760, 762, 764, and 766 couple receiver 770 and 710. However, lines 760, 762, 764 and 766 are coupled to terminals in receivers 770 and 710 which are not connected to other terminals within their respective receivers. That is, terminals 713, 723, and 733 are not coupled to any terminals within receiver 710; and terminals 771, 781, and 791 are not coupled to any terminals within receiver 770. In addition, line 766 couples terminal 731 of receiver 710 to terminal 792 of receiver 770. However, terminal 792 is not coupled to terminal 791, and terminal 731 is not coupled to terminal 732. As such, when a germicidal lamp is absent from both of receivers 710 and 770, no power is passed from receiver 710 to receiver 770. In addition, according to the interlock described herein, when a germicidal lamp having a connector is absent from one of receivers 710 and 770, the connections providing power to the remaining lamp in either receiver 710 to receiver 770 are removed. This is explained in more detail with regard to FIG. 8.

FIG. 8 is a block diagram of an embodiment of a completed interlock circuit formed when germicidal lamps are included in the mounting assemblies of a germicidal treatment system. That is, an interlock circuit is completed when lamps having coordinated connectors are placed in mounting assemblies having corresponding coordinated receivers. This drawing shows the result of placing lamps having connector 600 in receivers 710 and 770 shown in FIGS. 6 and 7.

In one embodiment, a germicidal lamp that includes a connector 600 requires power from terminals 613 and 633 to function. That is, when a germicidal lamp that includes a connector 600 is coupled in receiver 810, to function, the germicidal lamp obtains power from terminal 633 which corresponds to terminal 833 of receiver 810, and obtains power from terminal 613 which corresponds to terminal 833 of receiver 810. With regard to when a germicidal lamp that includes a connector 600 is coupled in receiver 870, to function, this germicidal lamp obtains power from terminal 633 which corresponds to terminal 891 of receiver 870, and obtains power from terminal 613 which corresponds to terminal 871 of receiver 870.

As discussed above, if no lamps are included in the receivers 810 and 870, when receiver 810 receives power from electrical power source 850, the electricity is not passed to receiver 870 because there is no connection between terminal 832 and 831. That is, power passing from power source 850 along line 852 to terminal 832 is not passed to terminal 831. Similarly, when receiver 810 receives power from electrical power source 850, the electricity is not passed to receiver 870 because there is no connection between terminal 812 and 813. That is, power passing from power source 850 along line 856 to terminal 812 is not passed to terminal 813.

When a coordinated connector is placed in receiver 810, a jumper completes the connection 843 between terminals 832 and 831 such that power is passed along line 866 to terminal 892 of receiver 870. Even though power has been provided to receiver 810 and connector 600 is placed in receive 810, no power is provided to terminal 833 through which the lamp is to receive power. In this way, a germicidal lamp having connector 600 placed in receiver 810 will not illuminate when the receiver is coupled to a power supply. That is, because a germicidal lamp having a connector is absent from receiver 870, no power is passed along line 864 from terminal 891 to terminal 833. Because of the interlock described herein, a germicidal lamp having connector 600 must also be placed in receiver 870 for power to be provided to a germicidal lamp in receiver 810.

When a germicidal lamp having connector 600 is placed in receiver 810, power is passed from terminal 832 to terminal 831. Power flows from terminal 831 over line 866 to terminal 892 of receiver 870. However, when no lamp is placed in receiver 870, no connection exists between terminal 892 and terminal 891. When a germicidal lamp having connector 600 is placed in receiver 870, a jumper completes a connection 873 between terminals 892 and 891. Concurrently, power is passed from terminal 891 along line 864 to terminal 833. As such, the germicidal lamps in each of receivers 810 and 870 are illuminated. Line 864 serves to return power from receiver 870 to receiver 810 that receiver 870 received via receiver 810. The activation or inactivation, the powering or not powering, of line 864 amounts to the interlock technique described herein.

In addition, when a germicidal lamp having a connector 600 is placed in receiver 810, a jumper completes connection 840 between terminals 812 and 813 such that current is passed over line 860 to terminal 871 in receiver 870. Further, when a germicidal lamp having a connector 600 is placed in receiver 810, a ground connection is formed when a jumper completes the connection 848 between terminals 822 and 823.

A full circuit is formed when lamps having connectors 600 are included in receivers 810 and 870 and receiver 810 is receiving energy from power source 850. If connector 600 is removed from receiver 870, the connection 873 is removed such that power no longer passes from terminal 891 across line 864. The lamp included in receiver 810 then ceases to receive power and ceases to emit UV radiation. Similarly, if connector 600 is removed from receiver 810, the connection 843 is removed such that power no longer passes from terminal 831 across line 866. The germicidal lamp included in receiver 870 then ceases to receive power and ceases to emit UV radiation. In this way, when a germicidal lamp of a dual lamp germicidal treatment system is removed from a mounting assembly, exposing a hole in a wall of an air handling apparatus, the removal of the germicidal lamp serves to immediately deactivate the other germicidal lamp in the other mounting assembly of the germicidal treatment system.

Although not shown in FIG. 8, in one embodiment, a ballast included in an integrated lamp only receives power when both germicidal lamps of a dual lamp germicidal treatment system are situated in mounting assemblies. According to the interlock described herein, when either of the germicidal lamps is removed from the germicidal treatment system, the interlock circuit is broken and no power is passed to the remaining integrated lamp. Therefore, no power is provided to the ballast in the remaining integrated lamp.

Similarly, although not shown in FIG. 8, in one embodiment, a ballast is included the mounting assemblies of a dual lamp germicidal treatment system. In this embodiment, the lamps are not integrated lamps. According to the interlock described herein, the ballasts in each of the mounting assemblies only receive power when both germicidal lamps of a dual lamp germicidal treatment system are situated in the mounting assemblies. When either of the germicidal lamps is removed from the germicidal treatment system, the interlock circuit is broken, no power is passed to either of the ballasts, and the remaining germicidal lamp is not irradiated.

In addition to the germicidal system embodiments described herein, the interlock technique and circuit may be employed with any number of electrically powered systems. For example, when a system has two or more units, each unit may include a receiver. For each unit, a device requiring power to function may be placed in the unit. The device may include a connector that corresponds to the receiver in the unit. The coordinated receivers/connectors couple the devices to the units. A single electrical power cord coupled to a first unit of the units. The units are coupled to one another such that when one or more of the devices are removed from the system, the electrical power to the other devices is terminated. That is, when one of the devices is removed from one of the units in the system, power is no longer delivered to the other units in the system. This is achieved using the interlock techniques described herein above.

Although exemplary embodiments of the invention have been shown and described, it will be apparent to those having ordinary skill in the art that a number of changes, modifications, or alterations to the invention as described herein may be made, none of which depart from the spirit of the invention. All such changes, modifications and alterations should therefore be seen as within the scope of the invention.

It is claimed:

1. A germicidal treatment system comprising:
a first mounting assembly having a first receiver
a second mounting assembly having a second receiver
a first germicidal lamp having a first connector to couple the first germicidal lamp to the first mounting assembly via the first receiver
a second germicidal lamp having a second connector to couple the second germicidal lamp to the second mounting assembly via the second receiver
a single electrical power cord coupled to the first mounting assembly to provide electrical power
the first mounting assembly adjustably coupled to the second mounting assembly such that the electrical power to the first germicidal lamp is terminated when the second germicidal lamp is removed from the second mounting assembly, and the electrical power to the second germicidal lamp will terminate when the first germicidal lamp is removed from the first mounting assembly.

2. The germicidal treatment system of claim 1 wherein the first germicidal lamp and the second germicidal lamp are the same kind of germicidal lamp such that the first connector and the second connector are the same kind of connector.

3. The germicidal treatment system of claim 1 wherein the first receiver and the second receiver have differing circuitry.

4. The germicidal treatment system of claim 1 wherein the first germicidal lamp has a first ballast incorporated therewith and the second germicidal lamp has a second ballast incorporated therewith.

5. The germicidal treatment system of claim 4 wherein the first receiver, the first connector, the second receiver, and the second connector are configured so no power is provided to either of the first ballast and the second ballast unless the first germicidal lamp is included in the first mounting assembly and the second germicidal lamp is included in the second mounting assembly.

6. The germicidal treatment system of claim 4 wherein the first mounting assembly has a first ballast incorporated therewith and the second mounting assembly has a second ballast incorporated therewith.

7. The germicidal treatment system of claim 6 wherein the first receiver, the first connector, the second receiver, and the second connector are configured so no power is provided to either of the first ballast and the second ballast unless the first germicidal lamp is included in the first mounting assembly and the second germicidal lamp is included in the second mounting assembly.

8. The germicidal treatment system of claim 1 further comprising a flexible cord connecting the first mounting assembly to the second mounting assembly.

9. The germicidal treatment system of claim 1 wherein the first mounting assembly and the second mounting assembly are situated on a first side of a wall of an air handling apparatus, and the first germicidal lamp and the second germicidal lamp are situated on a second side of the wall of the air handling apparatus.

10. The germicidal treatment system of claim 1 wherein the first receiver and the first connector are coordinated, and wherein the second receiver and the second connector are coordinated.

11. The germicidal treatment system of claim 1 wherein the first receiver and the first connector include coordinated male and female elements, wherein the second receiver and the second connector each include at least one half of coordinated male and female elements.

12. The germicidal treatment system of claim 111 wherein the first receiver and the second receiver have the same configuration.

13. An adjustable germicidal treatment system comprising:
two or more mounting assemblies each having a receiver
a germicidal lamp for each of the mounting assemblies, the germicidal lamp having a connector that couples the germicidal lamp to the mounting assemblies via the receiver
a single electrical power cord coupled to a first mounting assembly of the mounting assemblies
the mounting assemblies adjustably coupled one another and including circuitry such that when one or more of the germicidal lamps is removed from one of the mounting assemblies, power is not delivered to the remaining germicidal lamps.

14. The germicidal treatment system of claim 13 wherein the receiver of each mounting assembly includes differing circuitry from any neighboring mounting assemblies.

15. The germicidal treatment system of claim 13 wherein each of the germicidal lamps have a ballast incorporated therewith.

16. The germicidal treatment system of claim 15 wherein the receivers and the connectors are configured so no power is provided to the ballasts unless each of the germicidal lamp are included each of the mounting assemblies.

17. The germicidal treatment system of claim 13 wherein each of the mounting assemblies have a ballast incorporated therewith.

18. The germicidal treatment system of claim 13 further comprising a flexible cord connecting each of the mounting assemblies to neighbor mounting assemblies.

19. The germicidal treatment system of claim 13 wherein the mounting assemblies are situated on a first side of a wall of an air handling apparatus, and the germicidal lamps are situated on a second side of the wall of the air handling apparatus when the germicidal treatment system is attached to the air handling apparatus.

20. The germicidal treatment system of claim 13 wherein the receivers are coordinated with the connectors.

21. The germicidal treatment system of claim 13 wherein the receivers and the connectors each include at least one half of coordinated male and female elements.

22. An air treatment unit having the germicidal treatment system of claim 13 coupled thereto.

23. An air carrying duct having the adjustable germicidal treatment system of claim 13 coupled thereto.

24. An interlock circuit comprising:
two or more units each having a receiver
a device for each of the units, the device having a connector that couples the device to the units via the receiver
a single electrical power cord coupled to a first unit of the units
the units coupled to one another such that when one or more of the devices is removed from one of the units, power is not delivered to the remaining devices.

25. The interlock circuit of claim 24 wherein each of the receivers includes differing circuitry from any neighboring receivers.

26. The interlock circuit of claim 24 wherein the receivers and the connectors are configured so no power is provided to the devices unless each of the devices is included in each of the receivers.

27. The interlock circuit of claim 24 further comprising a flexible cord connecting each of the units to neighboring units.

28. The interlock circuit of claim 24 wherein the receivers are coordinated with the connectors.

29. The interlock circuit of claim 24 wherein the receivers and the connectors each include at least one half of coordinated male and female elements.
